(19) United States
(12) Patent Application Publication
(10) Pub. No.: US 2024/0194044 A1
(43) Pub. Date: Jun. 13, 2024

(54) ENVIRONMENTAL SAFETY DEVICE

(71) Applicant: Keonte Hemingway, Charlotte, NC (US)

(72) Inventor: Keonte Hemingway, Charlotte, NC (US)

(21) Appl. No.: 18/077,531

(22) Filed: Dec. 8, 2022

(51) Int. Cl. G08B 21/02; A61B 5/0205; G01K 1/024; G01K 3/00; G08B 7/06; G08B 21/18

(52) U.S. Cl. CPC G08B 21/02; A61B 5/0205; G01K 1/024; G01K 3/005; G08B 7/06; G08B 21/182; A61B 5/02438

(57) ABSTRACT

An environmental safety device, including a main body, a plurality of sensors disposed on at least a portion of the main body, the plurality of sensors including a heart rate sensor to monitor a heart rate of the user in response to contacting a portion of a body of a user, a temperature sensor to monitor a temperature level of an environment surrounding the temperature sensor, an air sensor to monitor an air quality level of the environment surrounding the air sensor, and a hydration sensor to monitor a dehydration level of the user in response to contacting a portion of the body of the user, a display unit disposed on at least a portion of the main body to display an alert in response to receiving an emergency signal, and a control unit disposed within at least a portion of the main body to transmit the emergency signal to the display unit in response to determining at least one of the heart rate exceeds a predetermined normal heart rate range, the temperature level exceeds a predetermined safe temperature range, the air quality level exceeds a predetermined normal air quality range, and the dehydration level falls below a predetermined normal hydration range.

### BACKGROUND

### 1. Field

The present general inventive concept relates generally to a safety device, and particularly, to an environmental safety device.

### 2. Description of the Related Art

For various reasons, some people are unknowingly exposed to unseen and/or unaware dangers. For example, a construction worker repairing a home and/or an office building may become hospitalized and/or injured due to exposure an area that has been contaminated with dangerous gases and/or extreme temperatures. Remaining in confined spaces that can quickly reach high, life-threatening temperatures can be deadly. Additionally, some areas can have air pollutants present that pose a serious threat on the health and safety of the construction worker who may not be cognizant of the impending danger.

Unfortunately, the construction worker is not provided any type of equipment to determine what potential hazards may exist in a given area. Thus, the construction worker is often injured without notice and discovery of the injury may be too late to reverse the damage.

Therefore, there is a need for an environmental safety device to detect any potential dangers within an environment and alert a user.

### SUMMARY

The present general inventive concept provides an environmental safety device.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing an environmental safety device, including a main body, a plurality of sensors disposed on at least a portion of the main body, the plurality of sensors including a heart rate sensor to monitor a heart rate of the user in response to contacting a portion of a body of a user, a temperature sensor to monitor a temperature level of an environment surrounding the temperature sensor, an air sensor to monitor an air quality level of the environment surrounding the air sensor, and a hydration sensor to monitor a dehydration level of the user in response to contacting a portion of the body of the user, a display unit disposed on at least a portion of the main body to display an alert in response to receiving an emergency signal, and a control unit disposed within at least a portion of the main body to transmit the emergency signal to the display unit in response to determining at least one of the heart rate exceeds a predetermined normal heart rate range, the temperature level exceeds a predetermined safe temperature range, the air quality level exceeds a predetermined normal air quality range, and the dehydration level falls below a predetermined normal hydration range.

The control unit may determine whether a change in at least one of the heart rate, the temperature level, the air quality level, and the dehydration level is based on a change in environment.

The display unit may emit a vocal warning to leave an area in response to receiving the emergency signal.

The air sensor may detect the air quality level based on a presence of at least one of at least one pathogen, dirt, and dust.

The air sensor may detect the air quality level based on the presence of COVID-19.

The control unit may transmit the emergency signal to the display unit based on the dehydration level of the user falling below ninety-five percent.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1A illustrates a rear perspective view of an environmental safety device, according to an exemplary embodiment of the present general inventive concept;

FIG. 1B illustrates a front perspective view of the environmental safety device, according to an exemplary embodiment of the present general inventive concept;

FIG. 2 illustrates a front perspective view of an environmental safety device, according to another exemplary embodiment of the present general inventive concept; and

FIG. 3 illustrates a side perspective view of an environmental safety device, according to another exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Environmental Safety Device 100
Main Body 110
Central Housing 111
First Strap 112
Second Strap 113
Buckle 114
Pin Receiving Apertures 115
Pin 116
Sensors 120
Heart Rate Sensor 121
Temperature Sensor 122
Air Sensor 123
Hydration Sensor 124
Display Unit 130
Control Unit 140
Input Button 150
Selection Dial 160
Power Source 170
Environmental Safety Device 200
Main Body 210
Sensors 220
Display Unit 230
Control Unit 240
Power Source 250
Environmental Safety Device 300
Main Body 310
Sensors 320
Display Unit 330
Control Unit 340
Power Source 350
Connection Strip 360

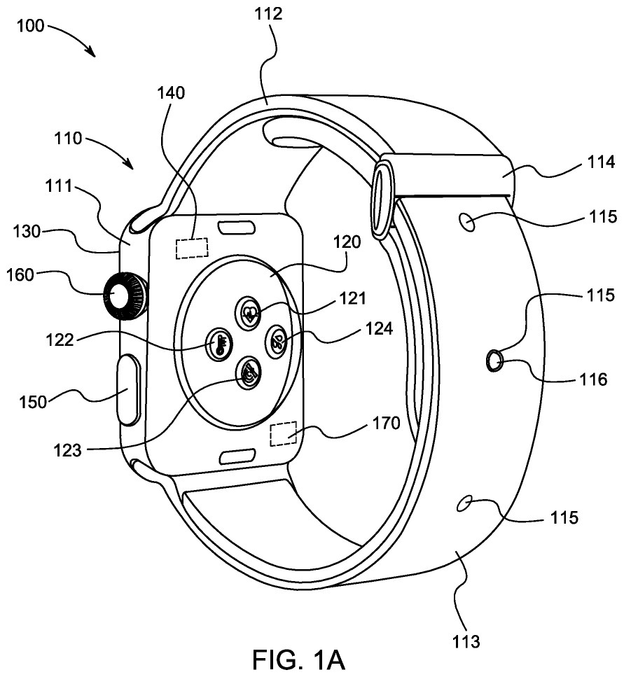

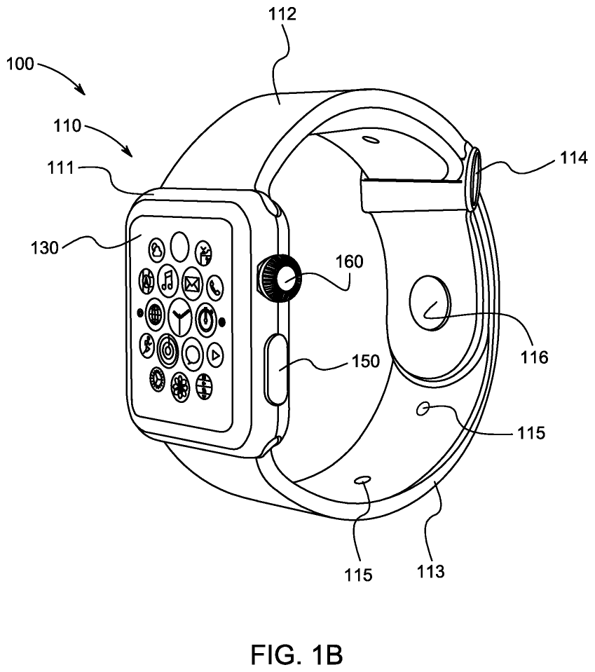

FIG. 1A illustrates a rear perspective view of an environmental safety device 100, according to an exemplary embodiment of the present general inventive concept.

FIG. 1B illustrates a front perspective view of the environmental safety device 100, according to an exemplary embodiment of the present general inventive concept.

The environmental safety device 100 may be constructed from at least one of metal, plastic, wood, glass, and rubber, etc., but is not limited thereto.

The environmental safety device 100 may include a main body 110, a plurality of sensors 120, a display unit 130, a control unit 140, an input button 150, a selection dial 160, and a power source 170, but is not limited thereto.

Referring to FIGS. 1A and 1B, the main body 110 is illustrated to be a watch. However, the main body 110 may be a bracelet, a necklace, a headband, a ring, or any other worn article and/or accessory known to one of ordinary skill in the art, but is not limited thereto.

The main body 110 may include a central housing 111, a first strap 112, a second strap 113, a buckle 114, a plurality of pin receiving apertures 115, and a pin 116, but is not limited thereto.

The central housing 111 may be removably connected to at least a portion of a body of a user, such as a wrist of the user. In other words, the central housing 111 may be worn on the body of the user.

The first strap 112 may be elongate and flexible. In other words, the first strap 112 may at least partially deform (e.g., bend, stretch) in response to an application of force (e.g., pushing, pulling) thereto. The first strap 112 may be disposed on at least a portion of the central housing 111. The first strap 112 may extend away from the central housing 111 in a first direction.

The second strap 113 may be elongate and flexible. In other words, the second strap 113 may at least partially deform (e.g., bend, stretch) in response to an application of force (e.g., pushing, pulling) thereto. The second strap 113 may be disposed on at least a portion of the central housing 111. The second strap 113 may extend away from the central housing 111 in a second direction opposite with respect to the first direction.

The buckle 114 may be disposed on at least a portion of the first strap 112. The buckle 114 may receive at least a portion of the second strap 113 therethrough. As such, the buckle 114 may removably connect the first strap 112 to the second strap 113. Thus, a size of a space between the central housing 111, the first strap 112, and/or the second strap 113 may be adjusted based on movement of the second strap 113 through the buckle 114.

The plurality of pin receiving apertures 115 may be disposed on at least a portion of the first strap 112 and/or the second strap 113.

The pin 116 may be disposed on at least a portion of the buckle 114. The pin 116 may be removably connected (i.e., inserted) into at least one of the plurality of pin receiving apertures 115. Thus, the pin 116 may prevent movement of the second strap 112 away from the buckle 114 while the pin 116 remains inserted within at least one of the plurality of pin receiving apertures 115.

The plurality of sensors 120 may include a heart rate sensor 121, a temperature sensor 122, an air sensor 123, and a hydration sensor 124, but is not limited thereto.

The heart rate sensor 121 may be disposed on at least a portion of a rear surface of the central housing 111. The heart rate sensor 121 may monitor and/or detect a heart rate of the user in response to contacting a portion of the body of the user (e.g., skin).

The temperature sensor 122 may be disposed on at least a portion of the rear surface of the central housing 111. The temperature sensor 122 may monitor and/or detect a temperature level of the user in response to contacting a portion of the body of the user (e.g., skin). Alternatively, the temperature sensor 122 may monitor and/or detect a temperature level of an environment surrounding the temperature sensor 122.

The air sensor 123 may be disposed on at least a portion of the rear surface of the central housing 111. The air sensor 123 may monitor and/or detect an air quality level of the environment surrounding the air sensor 123.

The hydration sensor 124 may be disposed on at least a portion of the rear surface of the central housing 111. The hydration sensor 124 may monitor and/or detect a dehydration level (e.g., percentage of dehydration level, milliliters of water) of the user in response to contacting a portion of the body of the user (e.g., skin). Alternatively, the hydration sensor 124 may monitor and/or detect a humidity level of the environment surrounding the hydration sensor 124.

The display unit 130 may include a plasma screen, an LCD screen, a light emitting diode (LED) screen, an organic LED (OLED) screen, a computer monitor, a hologram output unit, a sound outputting unit (e.g., a speaker), or any other type of device that visually or aurally displays data.

The display unit 130 may be disposed on at least a portion of the central housing 111. The display unit 130 may display an alert in response to receiving an emergency signal.

The control unit 140 may include a processing unit, a communication unit, and a storage unit, but is not limited thereto.

The processing unit of the control unit 140 (or central processing unit, CPU) may include electronic circuitry to carry out instructions of a computer program by performing basic arithmetic, logical, control and input/output (I/O) operations specified by the instructions. The processing unit of the control unit 140 may include an arithmetic logic unit (ALU) that performs arithmetic and logic operations, processor registers that supply operands to the ALU and store the results of ALU operations, and a control unit that fetches instructions from memory and “executes” them by directing the coordinated operations of the ALU, registers and other components. The processing unit of the control unit 140 may also include a microprocessor and a microcontroller.

The communication unit of the control unit 140 may include a device capable of wireless or wired communication between other wireless or wired devices via at least one of Wi-Fi, Wi-Fi Direct, infrared (IR) wireless communication, satellite communication, broadcast radio communication, Microwave radio communication, Bluetooth, Bluetooth Low Energy (BLE), Zigbee, near field communication (NFC), and radio frequency (RF) communication, USB, global positioning system (GPS), Firewire, and Ethernet.

The storage unit of the control unit 140 may include a random access memory (RAM), a read-only memory (ROM), a hard disk, a flash drive, a database connected to the Internet, cloud-based storage, Internet-based storage, or any other type of storage unit.

The control unit 140 may be disposed within at least a portion of the central housing 111. The control unit 140 may access the Internet via the communication unit to allow the user to access a website, and/or may allow a software application to be executed using the processing unit. For ease of description, the software application will be hereinafter referred to as an app. The app may be downloaded from the Internet to be stored on the storage unit.

The control unit 140 executing the app may receive data from the heart rate sensor 121, the temperature sensor 122, the air sensor 123, and/or the hydration sensor 124. Moreover, the control unit 140 executing the app may determine whether the data from the heart rate sensor 121, the temperature sensor 122, the air sensor 123, and/or the hydration sensor 124 is based on a presence of the user in a current environment. In other words, control unit 140 may determine whether a change in at least one of the heart rate, the temperature level, the air quality level, and the dehydration level is based on a change in environment.

The control unit 140 may compare and/or measure the heart rate of the user from the heart rate sensor 121 to a predetermined normal heart rate range (e.g., sixty to one hundred beats per minute). Subsequently, the control unit 140 may send the emergency signal to the display unit 130, such that the display unit 130 may display the alert and/or emit an audio warning (e.g., a ring, a beep, a siren, a voice message to “leave the area immediately” or “take a rest”) in response to the control unit 140 determining the heart rate exceeds the predetermined normal heart rate range.

Additionally, the control unit 140 may compare and/or measure the temperature level of the user from the temperature sensor 122 and/or the temperature level from the environment surrounding the temperature sensor 122 to a predetermined safe temperature range (e.g., above twenty degrees Fahrenheit, below ninety degrees Fahrenheit). Subsequently, the control unit 140 may send the emergency signal to the display unit 130, such that the display unit 130 may display the alert and/or emit the audio warning (e.g., a ring, a beep, a siren, a voice message to “leave the area immediately” or “cool down”) in response to the control unit 140 determining the temperature level exceeds the predetermined safe temperature range.

The control unit 140 may compare and/or measure the air quality level from the air sensor 123 to a predetermined normal air quality range (e.g., below one hundred air quality index, absence of dust, absence of dirt, lack of pathogens (e.g., bacteria, virus (e.g., coronavirus disease 2019, COVID-19), parasite, fungus), etc.). Subsequently, the control unit 140 may send the emergency signal to the display unit 130, such that the display unit 130 may display the alert and/or emit the audio warning (e.g., a ring, a beep, a siren, a voice message to “leave the area immediately” or “get some fresh air”) in response to the control unit 140 determining the air quality level exceeds the predetermined normal air quality range and/or detected presence of dust, dirt, and/or at least one pathogen.

Finally, the control unit 140 may compare and/or measure the dehydration level of the user from the hydration sensor 124 to a predetermined normal hydration range (e.g., above ninety-five percent hydration). Subsequently, the control unit 140 may send the emergency signal to the display unit 130, such that the display unit 130 may display the alert and/or emit the audio warning (e.g., a ring, a beep, a siren, a voice message to “leave the area immediately” or “get a drink of water”) in response to the control unit 140 determining the dehydration level falls below the predetermined normal hydration range.

Alternatively, the control unit 140 may compare and/or measure the humidity level of the environment from the hydration sensor 124 to a predetermined normal humidity range (e.g., below sixty percent humidity). Subsequently, the control unit 140 may send the emergency signal to the display unit 130, such that the display unit 130 may display the alert and/or emit the audio warning (e.g., a ring, a beep, a siren, a voice message to “leave the area immediately”) in response to the control unit 140 determining the humidity level exceeds the predetermined normal humidity range.

The control unit 140 may save a location based on a GPS position of a dangerous area, such that the control unit 140 may automatically and pre-emptively notify the user of the dangerous area prior to entry by the user while the user is within a predetermined distance (e.g., five feet, ten feet, twenty feet, etc.) of the dangerous area. For example, the control unit 140 may save the GPS position of a building determined to contain asbestos by the air sensor 123, such that the display unit 130 may warn the user of the danger of the building in response to control unit 140 determining the user is within fifteen feet of the building.

Furthermore, the control unit 140 may send the alert signal to at least one third party, such as a family member, a network support center, and/or a first responder (e.g., police, ambulance, fire and rescue, etc.). Moreover, the control unit 140 may broadcast a GPS signal to identify a location of the control unit 140, and therefore, a location of the user to receive assistance.

The input button 150 may be disposed on at least a portion of the central housing 111. The input button 150 may submit an entry on the display unit 130 for receipt by the control unit 140. However, the display unit 130 may be used to input the entry via a touchscreen. Also, the input button 150 may be used to cycle through selections for the plurality of sensors 120. In other words, the heart rate sensor 121, the temperature sensor 122, the air sensor 123, and/or the hydration sensor 124 may be turned on and/or turned off using the input button 150 and/or the display unit 130.

The selection dial 160 may be movably (i.e., rotatably) disposed on at least a portion of the central housing 111. The selection dial 160 may move (i.e., rotate) to select the entry on the display unit 130. For example, the selection dial 160 may be moved to highlight at least one of the plurality of sensors 120 to be used prior to entry via the input button 150. The selection dial 160 and/or the input unit 150 may be used to enter information, such as a name and/or a number of the at least one third party to contact during an emergency. Alternatively, the display unit 130 may be used to enter the same information.

The power source 170 may include a battery and a solar cell, but is not limited thereto.

The power source 170 may be disposed within at least a portion of the central housing 111. The power source 170 may provide power to the plurality of sensors 120, the display unit 130, the control unit 140, the input button 150, and/or the selection dial 160. The power source 170 may charge the battery in response to receiving power from an external light source via the solar cell.

Therefore, the environmental safety device 100 may save a life of the user entering a potentially dangerous environment. Also, the environmental safety device 100 may warn the user of the danger, such that the user may leave the dangerous environment.

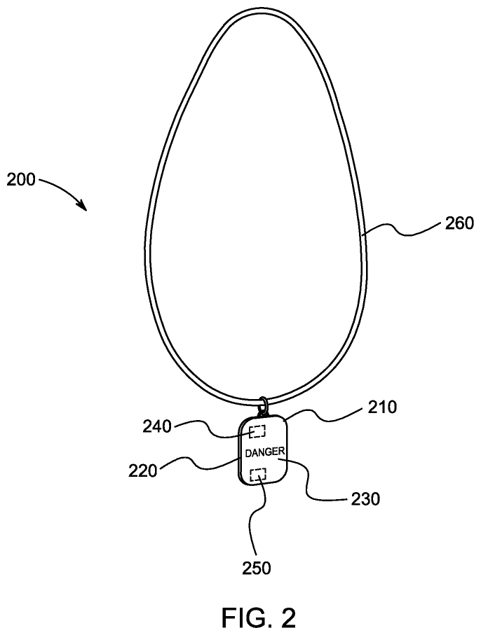

FIG. 2 illustrates a front perspective view of an environmental safety device 200, according to another exemplary embodiment of the present general inventive concept.

The environmental safety device 200 may be constructed from at least one of metal, plastic, wood, glass, and rubber, etc., but is not limited thereto.

The environmental safety device 200 may include a main body 210, a plurality of sensors 220, a display unit 230, a control unit 240, a power source 250, and a cord 260, but is not limited thereto.

Referring to FIG. 2, the main body 210 is illustrated to be a dongle. However, the main body 210 may be a lanyard, a necklace, a headband, a ring, or any other worn article and/or accessory known to one of ordinary skill in the art, but is not limited thereto. The main body 210 may be worn by the user.

The plurality of sensors 220 may include a heart rate sensor, a temperature sensor, an air sensor, and a hydration sensor, but is not limited thereto.

The heart rate sensor of the plurality of sensors 220 may be disposed on at least a portion of a rear surface of the main body 210. The heart rate sensor of the plurality of sensors 220 may monitor and/or detect a heart rate of the user in response to contacting a portion of the body of the user (e.g., skin).

The temperature sensor of the plurality of sensors 220 may be disposed on at least a portion of the rear surface of the main body 210. The temperature sensor of the plurality of sensors 220 may monitor and/or detect a temperature level of the user in response to contacting a portion of the body of the user (e.g., skin). Alternatively, the temperature sensor of the plurality of sensors 220 may monitor and/or detect a temperature level of an environment surrounding the temperature sensor of the plurality of sensors 220.

The air sensor of the plurality of sensors 220 may be disposed on at least a portion of the rear surface of the main body 210. The air sensor of the plurality of sensors 220 may monitor and/or detect an air quality level of the environment surrounding the air sensor of the plurality of sensors 220.

The hydration sensor of the plurality of sensors 220 may be disposed on at least a portion of the rear surface of the main body 210. The hydration sensor of the plurality of sensors 220 may monitor and/or detect a dehydration level (e.g., percentage of dehydration level, milliliters of water) of the user in response to contacting a portion of the body of the user (e.g., skin). Alternatively, the hydration sensor of the plurality of sensors 220 may monitor and/or detect a humidity level of the environment surrounding the hydration sensor of the plurality of sensors 220.

The display unit 230 may include a plasma screen, an LCD screen, a light emitting diode (LED) screen, an organic LED (OLED) screen, a computer monitor, a hologram output unit, a sound outputting unit (e.g., a speaker), or any other type of device that visually or aurally displays data.

The display unit 230 may be disposed on at least a portion of the main body 210. The display unit 230 may display an alert in response to receiving an emergency signal.

The control unit 240 may include a processing unit, a communication unit, and a storage unit, but is not limited thereto.

The processing unit of the control unit 240 (or central processing unit, CPU) may include electronic circuitry to carry out instructions of a computer program by performing basic arithmetic, logical, control and input/output (I/O) operations specified by the instructions. The processing unit of the control unit 240 may include an arithmetic logic unit (ALU) that performs arithmetic and logic operations, processor registers that supply operands to the ALU and store the results of ALU operations, and a control unit that fetches instructions from memory and “executes” them by directing the coordinated operations of the ALU, registers and other components. The processing unit of the control unit 240 may also include a microprocessor and a microcontroller.

The communication unit of the control unit 240 may include a device capable of wireless or wired communication between other wireless or wired devices via at least one of Wi-Fi, Wi-Fi Direct, infrared (IR) wireless communication, satellite communication, broadcast radio communication, Microwave radio communication, Bluetooth, Bluetooth Low Energy (BLE), Zigbee, near field communication (NFC), and radio frequency (RF) communication, USB, global positioning system (GPS), Firewire, and Ethernet.

The storage unit of the control unit 240 may include a random access memory (RAM), a read-only memory (ROM), a hard disk, a flash drive, a database connected to the Internet, cloud-based storage, Internet-based storage, or any other type of storage unit.

The control unit 240 may be disposed within at least a portion of the main body 210. The control unit 240 may access the Internet via the communication unit to allow the user to access a website, and/or may allow a software application to be executed using the processing unit. For ease of description, the software application will be hereinafter referred to as an app. The app may be downloaded from the Internet to be stored on the storage unit.

The control unit 240 executing the app may receive data from the heart rate sensor of the plurality of sensors 220, the temperature sensor of the plurality of sensors 220, the air sensor of the plurality of sensors 220, and/or the hydration sensor of the plurality of sensors 220. Moreover, the control unit 240 executing the app may determine whether the data from the heart rate sensor of the plurality of sensors 220, the temperature sensor of the plurality of sensors 220, the air sensor of the plurality of sensors 220, and/or the hydration sensor of the plurality of sensors 220 is based on a presence of the user in a current environment. In other words, the control unit 240 may determine whether a change in at least one of the heart rate, the temperature level, the air quality level, and the dehydration level is based on a change in environment.

The control unit 240 may compare and/or measure the heart rate of the user from the heart rate sensor of the plurality of sensors 220 to a predetermined normal heart rate range (e.g., sixty to one hundred beats per minute). Subsequently, the control unit 240 may send the emergency signal to the display unit 230, such that the display unit 230 may display the alert and/or emit an audio warning (e.g., a ring, a beep, a siren, a voice message to “leave the area immediately” or “take a rest”) in response to the control unit 240 determining the heart rate exceeds the predetermined normal heart rate range.

Additionally, the control unit 240 may compare and/or measure the temperature level of the user from the temperature sensor of the plurality of sensors 220 and/or the temperature level from the environment surrounding the temperature sensor of the plurality of sensors 220 to a predetermined safe temperature range (e.g., above twenty degrees Fahrenheit, below ninety degrees Fahrenheit). Subsequently, the control unit 240 may send the emergency signal to the display unit 230, such that the display unit 230 may display the alert and/or emit the audio warning (e.g., a ring, a beep, a siren, a voice message to “leave the area immediately” or “cool down”) in response to the control unit 240 determining the temperature level exceeds the predetermined safe temperature range.

The control unit 240 may compare and/or measure the air quality level from the air sensor of the plurality of sensors 220 to a predetermined normal air quality range (e.g., below one hundred air quality index, absence of dust, absence of dirt, lack of pathogens (e.g., bacteria, virus (e.g., COVID-19), parasite, fungus), etc.). Subsequently, the control unit 240 may send the emergency signal to the display unit 230, such that the display unit 230 may display the alert and/or emit the audio warning (e.g., a ring, a beep, a siren, a voice message to “leave the area immediately” or “get some fresh air”) in response to the control unit 240 determining the air quality level exceeds the predetermined normal air quality range and/or detected presence of dust, dirt, and/or at least one pathogen.

Finally, the control unit 240 may compare and/or measure the dehydration level of the user from the hydration sensor of the plurality of sensors 220 to a predetermined normal hydration range (e.g., above ninety-five percent hydration). Subsequently, the control unit 240 may send the emergency signal to the display unit 230, such that the display unit 230 may display the alert and/or emit the audio warning (e.g., a ring, a beep, a siren, a voice message to “leave the area immediately” or “get a drink of water”) in response to the control unit 240 determining the dehydration level falls below the predetermined normal hydration range.

Alternatively, the control unit 240 may compare and/or measure the humidity level of the environment from the hydration sensor of the plurality of sensors 220 to a predetermined normal humidity range (e.g., below sixty percent humidity). Subsequently, the control unit 240 may send the emergency signal to the display unit 230, such that the display unit 230 may display the alert and/or emit the audio warning (e.g., a ring, a beep, a siren, a voice message to “leave the area immediately”) in response to the control unit 240 determining the humidity level exceeds the predetermined normal humidity range.

The control unit 240 may save a location based on a GPS position of a dangerous area, such that the control unit 240 may automatically and pre-emptively notify the user of the dangerous area prior to entry by the user while the user is within a predetermined distance (e.g., five feet, ten feet, twenty feet, etc.) of the dangerous area. For example, the control unit 240 may save the GPS position of a building determined to contain asbestos by the air sensor of the plurality of sensors 220, such that the display unit 230 may warn the user of the danger of the building in response to control unit 240 determining the user is within fifteen feet of the building.

Furthermore, the control unit 240 may send the alert signal to at least one third party, such as a family member, a network support center, and/or a first responder (e.g., police, ambulance, fire and rescue, etc.). Moreover, the control unit 240 may broadcast a GPS signal to identify a location of the control unit 240, and therefore, a location of the user to receive assistance.

The power source 250 may include a battery and a solar cell, but is not limited thereto.

The power source 250 may be disposed within at least a portion of the main body 210. The power source 250 may provide power to the plurality of sensors 220, the display unit 230, and/or the control unit 240. The power source 250 may charge the battery in response to receiving power from an external light source via the solar cell.

The cord 260 may be disposed on at least a portion of the main body 210. The cord 260 may be elastic. As such, the cord 260 may at least partially deform (e.g., bend, stretch, expand) in response to an application of force (e.g., pushing, pulling) thereto. The cord 260 may be worn by the user, such as around a neck of the user. Accordingly, the main body 210 may be suspended from the cord 260 while being worn by the user.

Therefore, the environmental safety device 200 may save a life of the user entering a potentially dangerous environment. Also, the environmental safety device 200 may warn the user of the danger, such that the user may leave the dangerous environment.

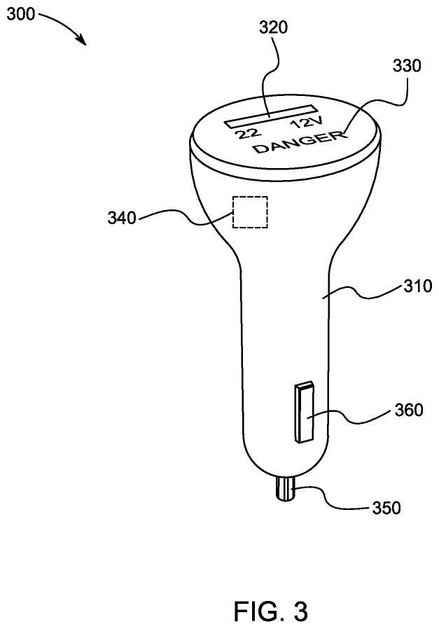

FIG. 3 illustrates a side perspective view of an environmental safety device 300, according to another exemplary embodiment of the present general inventive concept.

The environmental safety device 300 may be constructed from at least one of metal, plastic, wood, glass, and rubber, etc., but is not limited thereto.

The environmental safety device 300 may include a main body 310, a plurality of sensors 320, a display unit 330, a control unit 340, a power source 350, and a connecting surface 360, but is not limited thereto.

Referring to FIG. 3, the main body 310 is illustrated to be a vehicle cigarette adapter. However, the main body 310 may be a switch, a knob, a universal serial bus (USB) device, or any other vehicle connecting device and/or accessory known to one of ordinary skill in the art, but is not limited thereto.

The plurality of sensors 320 may include a heart rate sensor, a temperature sensor, an air sensor, and a hydration sensor, but is not limited thereto.

The heart rate sensor of the plurality of sensors 320 may be disposed on at least a portion of a rear surface of the main body 310. The heart rate sensor of the plurality of sensors 320 may monitor and/or detect a heart rate of the user in response to contacting a portion of the body of the user (e.g., skin).

The temperature sensor of the plurality of sensors 320 may be disposed on at least a portion of the rear surface of the main body 310. The temperature sensor of the plurality of sensors 320 may monitor and/or detect a temperature level of the user in response to contacting a portion of the body of the user (e.g., skin). Alternatively, the temperature sensor of the plurality of sensors 320 may monitor and/or detect a temperature level of an environment surrounding the temperature sensor of the plurality of sensors 320.

The air sensor of the plurality of sensors 320 may be disposed on at least a portion of the rear surface of the main body 310. The air sensor of the plurality of sensors 320 may monitor and/or detect an air quality level of the environment surrounding the air sensor of the plurality of sensors 320.

The hydration sensor of the plurality of sensors 320 may be disposed on at least a portion of the rear surface of the main body 310. The hydration sensor of the plurality of sensors 320 may monitor and/or detect a dehydration level (e.g., percentage of dehydration level, milliliters of water) of the user in response to contacting a portion of the body of the user (e.g., skin). Alternatively, the hydration sensor of the plurality of sensors 320 may monitor and/or detect a humidity level of the environment surrounding the hydration sensor of the plurality of sensors 320.

The display unit 330 may include a plasma screen, an LCD screen, a light emitting diode (LED) screen, an organic LED (OLED) screen, a computer monitor, a hologram output unit, a sound outputting unit (e.g., a speaker), or any other type of device that visually or aurally displays data.

The display unit 330 may be disposed on at least a portion of the main body 310. The display unit 330 may display an alert in response to receiving an emergency signal.

The control unit 340 may include a processing unit, a communication unit, and a storage unit, but is not limited thereto.

The processing unit of the control unit 340 (or central processing unit, CPU) may include electronic circuitry to carry out instructions of a computer program by performing basic arithmetic, logical, control and input/output (I/O) operations specified by the instructions. The processing unit of the control unit 340 may include an arithmetic logic unit (ALU) that performs arithmetic and logic operations, processor registers that supply operands to the ALU and store the results of ALU operations, and a control unit that fetches instructions from memory and “executes” them by directing the coordinated operations of the ALU, registers and other components. The processing unit of the control unit 340 may also include a microprocessor and a microcontroller.

The communication unit of the control unit 340 may include a device capable of wireless or wired communication between other wireless or wired devices via at least one of Wi-Fi, Wi-Fi Direct, infrared (IR) wireless communication, satellite communication, broadcast radio communication, Microwave radio communication, Bluetooth, Bluetooth Low Energy (BLE), Zigbee, near field communication (NFC), and radio frequency (RF) communication, USB, global positioning system (GPS), Firewire, and Ethernet.

The storage unit of the control unit 340 may include a random access memory (RAM), a read-only memory (ROM), a hard disk, a flash drive, a database connected to the Internet, cloud-based storage, Internet-based storage, or any other type of storage unit.

The control unit 340 may be disposed within at least a portion of the main body 310. The control unit 340 may access the Internet via the communication unit to allow the user to access a website, and/or may allow a software application to be executed using the processing unit. For ease of description, the software application will be hereinafter referred to as an app. The app may be downloaded from the Internet to be stored on the storage unit.

The control unit 340 executing the app may receive data from the heart rate sensor of the plurality of sensors 320, the temperature sensor of the plurality of sensors 320, the air sensor of the plurality of sensors 320, and/or the hydration sensor of the plurality of sensors 320. Moreover, the control unit 340 executing the app may determine whether the data from the heart rate sensor of the plurality of sensors 320, the temperature sensor of the plurality of sensors 320, the air sensor of the plurality of sensors 320, and/or the hydration sensor of the plurality of sensors 320 is based on a presence of the user in a current environment. In other words, the control unit 340 may determine whether a change in at least one of the heart rate, the temperature level, the air quality level, and the dehydration level is based on a change in environment.

The control unit 340 may compare and/or measure the heart rate of the user from the heart rate sensor of the plurality of sensors 320 to a predetermined normal heart rate range (e.g., sixty to one hundred beats per minute). Subsequently, the control unit 340 may send the emergency signal to the display unit 330, such that the display unit 330 may display the alert and/or emit an audio warning (e.g., a ring, a beep, a siren, a voice message to “leave the area immediately” or “take a rest”) in response to the control unit 340 determining the heart rate exceeds the predetermined normal heart rate range.

Additionally, the control unit 340 may compare and/or measure the temperature level of the user from the temperature sensor of the plurality of sensors 320 and/or the temperature level from the environment surrounding the temperature sensor of the plurality of sensors 320 to a predetermined safe temperature range (e.g., above twenty degrees Fahrenheit, below ninety degrees Fahrenheit). Subsequently, the control unit 340 may send the emergency signal to the display unit 230, such that the display unit 330 may display the alert and/or emit the audio warning (e.g., a ring, a beep, a siren, a voice message to “leave the area immediately” or “cool down”) in response to the control unit 340 determining the temperature level exceeds the predetermined safe temperature range.

The control unit 340 may compare and/or measure the air quality level from the air sensor of the plurality of sensors 320 to a predetermined normal air quality range (e.g., below one hundred air quality index, absence of dust, absence of dirt, lack of pathogens (e.g., bacteria, virus (e.g., COVID-19), parasite, fungus), etc.). Subsequently, the control unit 340 may send the emergency signal to the display unit 330, such that the display unit 330 may display the alert and/or emit the audio warning (e.g., a ring, a beep, a siren, a voice message to “leave the area immediately” or “get some fresh air”) in response to the control unit 340 determining the air quality level exceeds the predetermined normal air quality range and/or detected presence of dust, dirt, and/or at least one pathogen.

Finally, the control unit 340 may compare and/or measure the dehydration level of the user from the hydration sensor of the plurality of sensors 320 to a predetermined normal hydration range (e.g., above ninety-five percent hydration). Subsequently, the control unit 340 may send the emergency signal to the display unit 330, such that the display unit 330 may display the alert and/or emit the audio warning (e.g., a ring, a beep, a siren, a voice message to “leave the area immediately” or “get a drink of water”) in response to the control unit 340 determining the dehydration level falls below the predetermined normal hydration range.

Alternatively, the control unit 340 may compare and/or measure the humidity level of the environment from the hydration sensor of the plurality of sensors 320 to a predetermined normal humidity range (e.g., below sixty percent humidity). Subsequently, the control unit 340 may send the emergency signal to the display unit 330, such that the display unit 330 may display the alert and/or emit the audio warning (e.g., a ring, a beep, a siren, a voice message to “leave the area immediately”) in response to the control unit 340 determining the humidity level exceeds the predetermined normal humidity range.

The control unit 340 may save a location based on a GPS position of a dangerous area, such that the control unit 340 may automatically and pre-emptively notify the user of the dangerous area prior to entry by the user while the user is within a predetermined distance (e.g., five feet, ten feet, twenty feet, etc.) of the dangerous area. For example, the control unit 340 may save the GPS position of a building determined to contain asbestos by the air sensor of the plurality of sensors 320, such that the display unit 330 may warn the user of the danger of the building in response to control unit 340 determining the user is within fifteen feet of the building.

Furthermore, the control unit 340 may send the alert signal to at least one third party, such as a family member, a network support center, and/or a first responder (e.g., police, ambulance, fire and rescue, etc.). Moreover, the control unit 340 may broadcast a GPS signal to identify a location of the control unit 340, and therefore, a location of the user to receive assistance.

The power source 350 may include an automobile auxiliary power outlet plug, but is not limited thereto.

The power source 350 may be disposed within at least a portion of the main body 310. The power source 350 may provide power to the plurality of sensors 320, the display unit 330, and/or the control unit 240 in response to connecting an external power source (e.g., an automobile auxiliary power outlet).

The connecting surface 360 may be disposed on at least a portion of the main body 310 and/or connected to the power source 350. The connecting surface 360 may connect to the external power source and/or prevent the main body 110 from moving away from (i.e., falling out of) the external power source.

Therefore, the environmental safety device 300 may save a life of the user entering a potentially dangerous environment. Also, the environmental safety device 300 may warn the user of the danger, such that the user may leave the dangerous environment.

The present general inventive concept may include an environmental safety device 100, including a main body 110, a plurality of sensors 120 disposed on at least a portion of the main body 110, the plurality of sensors 120 including a heart rate sensor 121 to monitor a heart rate of the user in response to contacting a portion of a body of a user, a temperature sensor 122 to monitor a temperature level of an environment surrounding the temperature sensor 122, an air sensor 123 to monitor an air quality level of the environment surrounding the air sensor 123, and a hydration sensor 124 to monitor a dehydration level of the user in response to contacting a portion of the body of the user, a display unit 130 disposed on at least a portion of the main body 110 to display an alert in response to receiving an emergency signal, and a control unit 140 disposed within at least a portion of the main body 110 to transmit the emergency signal to the display unit 130 in response to determining at least one of the heart rate exceeds a predetermined normal heart rate range, the temperature level exceeds a predetermined safe temperature range, the air quality level exceeds a predetermined normal air quality range, and the dehydration level falls below a predetermined normal hydration range.

The control unit 140 may determine whether a change in at least one of the heart rate, the temperature level, the air quality level, and the dehydration level is based on a change in environment.

The display unit 130 may emit a vocal warning to leave an area in response to receiving the emergency signal.

The air sensor 123 may detect the air quality level based on a presence of at least one of at least one pathogen, dirt, and dust.

The air sensor 123 may detect the air quality level based on the presence of COVID-19.

The control unit 140 may transmit the emergency signal to the display unit 130 based on the dehydration level of the user falling below ninety-five percent.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. An environmental safety device, comprising:
a main body;
a plurality of sensors disposed on at least a portion of the main body, the plurality of sensors comprising:
a heart rate sensor to monitor a heart rate of the user in response to contacting a portion of a body of a user,
a temperature sensor to monitor a temperature level of an environment surrounding the temperature sensor,
an air sensor to monitor an air quality level of the environment surrounding the air sensor, and
a hydration sensor to monitor a dehydration level of the user in response to contacting a portion of the body of the user;
a display unit disposed on at least a portion of the main body to display an alert in response to receiving an emergency signal; and
a control unit disposed within at least a portion of the main body to transmit the emergency signal to the display unit in response to determining at least one of the heart rate exceeds a predetermined normal heart rate range, the temperature level exceeds a predetermined safe temperature range, the air quality level exceeds a predetermined normal air quality range, and the dehydration level falls below a predetermined normal hydration range.

2. The environmental safety device of claim 1, wherein the control unit determines whether a change in at least one of the heart rate, the temperature level, the air quality level, and the dehydration level is based on a change in environment.

3. The environmental safety device of claim 1, wherein the display unit emits a vocal warning to leave an area in response to receiving the emergency signal.

4. The environmental safety device of claim 1, wherein the air sensor detects the air quality level based on a presence of at least one of at least one pathogen, dirt, and dust.

5. The environmental safety device of claim 4, wherein the air sensor detects the air quality level based on the presence of COVID-19.

6. The environmental safety device of claim 1, wherein the control unit transmits the emergency signal to the display unit based on the dehydration level of the user falling below ninety-five percent.
